(19) United States
(12) Patent Application Publication
(10) Pub. No.: US 2018/0325390 A1
(43) Pub. Date: Nov. 15, 2018

(54) BIOMETRIC DATA ANALYSIS

(71) Applicant: HEWLETT-PACKARD DEVELOPMENT COMPANY, LP., Houston, TX (US)

(72) Inventors: Matthew Flach, Fort Collins, CO (US); Amol Subhash Pandit, Fort Collins, CO (US); Peter Andrew Seiler, Fort Collins, CO (US)

(73) Assignee: HEWLETT-PACKARD DEVELOPMENT COMPANY, LP., Houston, TX (US)

(21) Appl. No.: 15/772,445

(22) PCT Filed: Feb. 10, 2016
(86) PCT No.: PCT/US2016/017260

§ 371 Date: Apr. 30, 2018

(51) Int. Cl. A61B 5/0205; G16H 10/60; A61B 5/0255; A61B 5/024; A61B 5/00; A61B 5/11

(52) U.S. Cl. CPC A61B 5/02055; G16H 10/60; A61B 5/0255; A61B 2562/0219; A61B 5/442; A61B 5/1107; A61B 2562/0271; A61B 5/02438

(57) ABSTRACT

An example system, for analyzing biometric data, is disclosed. The system includes a sensor unit to measure biometric data of a user. The system also includes a controller unit coupled to the sensor to perform an analysis of the biometric data based on at least one predetermined criteria and determine at least one setting adjustment for a device based on the analysis. Further, the system includes a communication unit to deliver an instruction from the controller to the device. The instruction includes the at least one setting adjustment associated with the device.

### BACKGROUND

Biometrics refers to metrics related to human characteristics. Biometric data defines one or more physical or behavioral characteristics of the individual and includes signature, voice and fingerprint data and the like. Biometric identifiers are the distinctive, measurable characteristics used to label and describe individuals. Biometric identifiers are often categorized as physiological versus behavioral characteristics. Physiological characteristics are related to the shape of the body. Examples include, but are not limited to fingerprint, palm veins, face recognition, DNA, palm print, hand geometry, iris recognition, retina and odor/scent. Behavioral characteristics are related to the pattern of behavior of a person, including but not limited to typing rhythm, gait, and voice. Biometric data may be used to verify or authenticate an individual prior to permitting that individual to take part in a transfer of value transaction, gain access to a building and the like.

### BRIEF DESCRIPTION OF THE DRAWINGS

For a detailed description of various examples, reference will now be made to the accompanying drawings in which:

FIG. 1 is a block diagram of an example system in accordance with the principles disclosed herein;

FIG. 2 is a schematic view of the system of FIG. 1 in accordance with the principles disclosed herein; and

FIG. 3 is a flowchart of an example method executable by a system of FIG. 1 in accordance with the principles disclosed herein.

### NOTATION AND NOMENCLATURE

Certain terms are used throughout the following description and claims to refer to particular system components. As one skilled in the art will appreciate, computer companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct electrical or mechanical connection, through an indirect electrical or mechanical connection via other devices and connections, through an optical electrical connection, or through a wireless electrical connection. As used herein the term “approximately” means plus or minus 10%. In addition, as used herein, the phrase “user input device” refers to any suitable device for providing an input, by a user, into an electrical system such as, for example, a mouse, keyboard, a hand (or any finger thereof), a stylus, a pointing device, etc.

### DETAILED DESCRIPTION

The following discussion is directed to various examples of the disclosure. Although one or more of these examples may be preferred, the examples disclosed should not be interpreted, or otherwise used, as limiting the scope of the disclosure, including the claims. In addition, one skilled in the art will understand that the following description has broad application, and the discussion of any example is meant only to be descriptive of that example, and not intended to intimate that the scope of the disclosure, including the claims, is limited to that example.

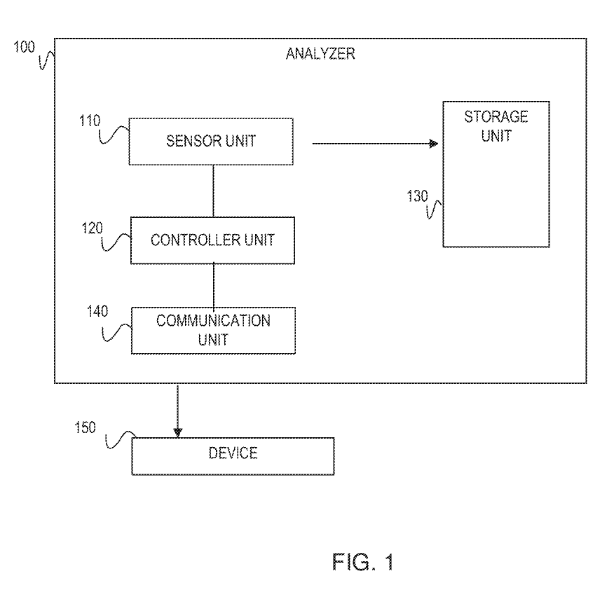

Referring now to FIG. 1, a biometric data analyzer 100 in accordance with the principles disclosed herein is shown. In this example, analyzer 100 generally comprises a sensor unit 110, a control unit 120, a storage unit 130 and a communications unit 140. In some implementations, the analyzer 100 can be a stand-alone device (which is shown in FIG. 1). Further, the analyzer 100 may be portable (e.g., mobile device, tablet, smart watch and alike). For example, the user may choose to use the analyzer 100 as a wearable accessory. More specifically, the user may choose to carry the system around in an environment (e.g., fitness center, home, office). In other implementations, the analyzer 100 may be positioned in another electronic device, such a smartphone, a tablet, a phablet, a smart watch or some combination thereof.

The analyzer 100 is communicatively connected to device 150. The device 150 may comprise any suitable device while still complying with the principles disclosed herein. For example, in some implementations, the device 150 may be a treadmill, kiosk, point-of-sale fixture, robot, or any other similar system containing a compute device. Further, the device 150 may have a permanent location in an environment (e.g., a room in a house), In other examples, the device 150 may be a portable device that is attached to the analyzer 100 and that moves with the user. In either example, the analyzer 100 maintains connection with the device 150. When the operation of the analyzer 100 is initiated, the analyzer 100 may confirm active connection with the device 150, collect biometric data of a user, analyze such data based on predetermined criteria and instruct the device 150 to adjust settings.

Further, in an alternative implementation, the analyzer 100 may comprise a display unit. In such example, the biometric data may be presented on the display unit. In other implementations, the display unit may be in the device 150. For example, a user of the analyzer 100 may use the display unit in the device 150 to interact with the analyzer 100. In another example, the display unit may be touch sensitive, and the user can use touch gestures to interact with the analyzer 100 or the device 150. Further, the display unit may be a transparent liquid crystal display (LCD), an organic light emitting diode (OLED) display, a plasma display, or any other suitable display,

The sensor 110 in the analyzer 100 collects biometric data of a user of the analyzer 100. The sensor unit 110 may include any suitable biometric sensor configured to measure one or more of but is not limited to, heart rate, pulse rate, temperature, respiration, acceleration, skin resistivity, muscle contractions, and/or alike. In another implementation, more than one sensor unit 110 may be provided. In one implementation, the sensor 110 produces a data signal that is indicative of one or more biometric parameters. As mentioned earlier, the biometric parameters may comprise, but not limited to, the user's heart rate, temperature and/or alike. According to an example of the present invention, biometric attributes such as pulse and body temperature can be monitored and stored in storage unit 130. Examples of storage units may include, but are not limited to, hard disk drives, compact disc drives, digital versatile disc drives, optical devices, and flash memory devices. In the present implementation, the storage unit 130 is shown in the analyzer 100. In another implementation, the storage unit 130 may be in the device 150, and the user's biometric data may be stored in the device 150. In a further implementation, the biometric data may be stored in the cloud. Further, the stored data may be used to create a profile for the user associated with that data. For example, the user's temperature at different times of the day may be recorded and saved under his profile. The analyzer 100 may access the profile and perform analysis of data under that profile in view of new data being collected by the analyzer 100. In other examples, the user profile may include user data such as user preferences, user settings, prescribed targets, historical biometric data, and/or alike. User preferences may include a user's workout schedule or workout intensity. User settings may include user's selection of a certain interface on the device. Prescribed targets may include target biometric levels prescribed for the user by a doctor or any other medical professional or recommended by a personal trainer. It should be noted that though a few examples are listed here for profile information, a user's profile may contain other types of data related to the user, general trends or the overall industry in addition to data related to location and time.

The sensor unit 110 is coupled to the controller unit 120. The controller unit 120 may include a programmable logic controller, microprocessor, application specific integrated circuit, or the like having suitable programming code for performing the methods described herein. More specifically, the controller unit may be implemented using any suitable type of processing system where at least one processor executes computer-readable instructions stored in a memory. The processor may be, for example, a central processing unit (CPU), a semiconductor-based microprocessor, an application specific integrated circuit (ASIC), a field-programmable gate array (FPGA) configured to retrieve and execute instructions, other electronic circuitry suitable for the retrieval and execution instructions stored on a computer readable storage medium (e.g., the memory), or a combination thereof. The computer readable medium may be a non-transitory computer-readable medium that stores machine readable instructions, codes, data, and/or other information, The instructions, when executed by the processor (e.g., via one processing element or multiple processing elements of the processor) can cause the processor to perform processes described herein. The computer readable medium may be one or more of a non-volatile memory, a volatile memory, and/or one or more storage devices. Examples of non-volatile memory include, but are not limited to, electronically erasable programmable read only memory (EEPROM) and read only memory (ROM). Examples of volatile memory include, but are not limited to, static random access memory (SRAM) and dynamic random access memory (DRAM).

The controller unit 120 analyzes the biometric data measured by the sensor unit 110 based on a predetermined criteria, In one implementation, such predetermined criteria may comprise historical data, trend data, and/or user preferences, and may be saved under the user's profile. The analyzer may automatically apply the predetermined criteria to the measured biometric data by accessing that information through the user profile, In another implementation, the predetermined criteria may be provided to the analyzer 100 by the user. In such example, the analyzer prompts the user to enter information regarding the criteria. In one example, during the analysis phase, the controller 120 may determine a change in the biometric data based on the user's profile (e.g., historical data). Further, during the analysis phase, the controller 120 determines at least one device setting change for the device 150 based on the biometric data or the change in the biometric data. Moreover, the controller unit 120 instructs the device 150 to implement the at least one change. In some implementations, more than one controller unit 120 may be used.

Further, the analyzer 100 comprises the communications unit 140 such as a transmitter/receiver chip that allows the controller unit 120 to communicate with the device 150 or another computing device (e.g., laptop, tablet). In some embodiments, the controller unit 120 may send information to and/or receive from the device 150 through any suitable type of connection while still complying with the principles disclosed herein. For example, in some implementations, the analyzer 100 is electrically coupled to the device 150 through an electric conductor, WI-FI, BLUETOOTH®, WiGig, an optical connection, an ultrasonic connection, or some combination thereof. Any suitable wireless (or wired electrical coupling) connection may be used between the analyzer 100 and the device 150 (if they are not in one unit) such as, for example, WI-FI, BLUETOOTH®, ultrasonic, electrical cables, electrical leads, electrical spring-loaded pogo pins with magnetic holding force, or some combination thereof, while still complying with the principles disclosed herein.

In some implementations, a battery (not shown in FIG. 1) may be provided for powering sensor unit 110, controller unit 120 and/or all the other components. In some embodiments, the battery may be rechargeable and/or replaceable. In one particular embodiment, the battery may be recharged via a USB connection (not shown in FIG. 1) that allows information to be transferred to another device such as a computer (not shown in FIG. 1).

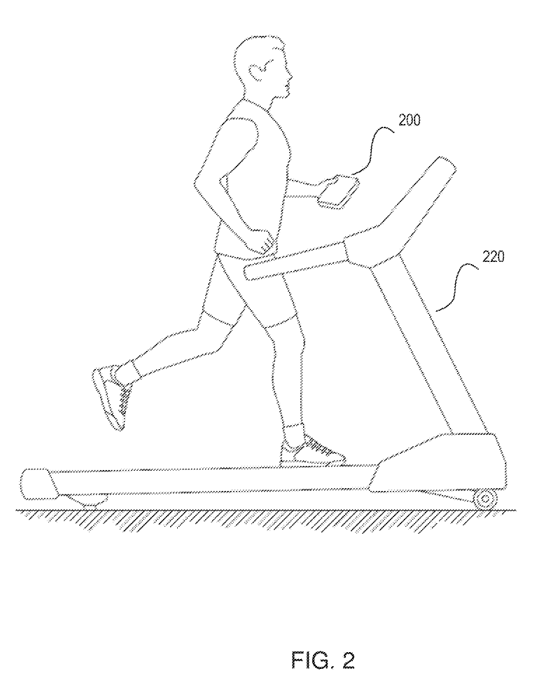

Referring now to FIG. 2, an analyzer 200 in accordance with the principles disclosed herein is shown. Similar to the analyzer 100 discussed in reference to FIG. 1, the analyzer 200 is attached to a device. In this example, the device is a treadmill 220. In this implementation, the analyzer 200 is shown as a portable rectangular box. A sensor and controller are substantially hidden inside the housing unit (rectangular box). In other implementations, the housing unit may be any suitable structure for supporting the components while still complying with the principles disclosed herein. The analyzer 200 may come in any shape and size, and can be position anywhere on or inside the user's body. In one implementation, the user may be holding the analyzer 200 (as shown in FIG. 2). In another implementation, the analyzer 200 may be placed on the body of the user as a wearable device (e.g., watch, necklace, bracelet, band, and earpiece). In a further implementation, the analyzer 200 may be physically attached to or implanted inside of the user, such as an implanted medical device or other form of biometric collection device.

As described in detail in reference to FIG. 1, the analyzer may measure certain biometric data associated with the user. For example, the analyzer 200 may measure the heart rate of a user. Based on the measured heart rate data, the controller in the analyzer 200 may instruct the treadmill 220 to adjust its settings. Heart rate monitoring (“HRM”) provides one way to determine and track the intensity of an exercise routine or workout session. An individual can maintain a desired intensity level by monitoring their heart rate during exercise using a heart rate monitor (HRM), Monitoring certain exercise metrics including the intensity and duration of an exercise session can provide an individual with useful information that may be used to establish and monitor fitness goals. The analyzer 200 handles this role for the user.

In addition, as will explained in more detail below, the sensor within the analyzer is communicatively coupled to the controller in the analyzer such that data generated within the sensor may be transmitted to the controller and commands issued by the controller may be communicated to the treadmill 220 during operations. As is explained above, any suitable electrical and/or communicative coupling may be used to couple analyzer to the device such as for example, an electric conductor, WI-FI, WiGig, BLUETOOTH®, an optical connection, an ultrasonic connection, or some combination thereof.

It should be noted that in other implementations, the analyzer 200 may be utilized for many other areas including, commerce (e.g., shopping), education, film, gaming, healthcare, fashion and alike. Other examples may be provided while still complying with the principles disclosed herein.

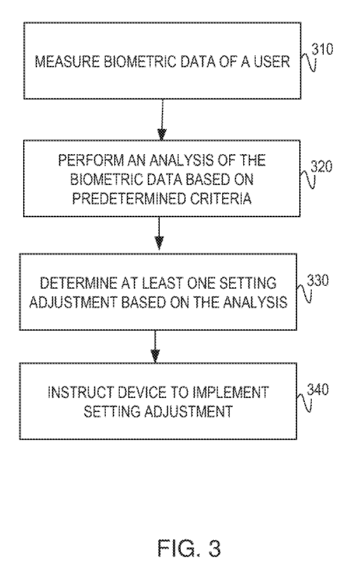

Referring now to FIG. 3, a flowchart of an example method executable by a system similar to the analyzers 100-200 described in reference to FIGS. 1-2 is shown in accordance with the principles disclosed herein. At block 310, the sensor in the analyzer measures biometric data of a user. As mentioned before, the biometric data may comprise heart rate, pulse rate, temperature, respiration, acceleration, skin resistivity, muscle contractions and/or alike. In one implementation, the measured biometric data may be stored in a storage unit. The storage unit may be located in the analyzer or in a device that the analyzer is communicatively connected to. Further, the biometric data may be saved under a profile associated with the user. In one implementation, the profile information may be accessed by the analyzer. In such an implementation, accessing the profile may require a username and password or other form of secure access

At block 320, the analyzer analyzes the biometric data based on a predetermined criteria, In one example, the biometric data may be body temperature, and the device may be a treadmill. Further, the predetermined criteria may comprise the user's historical performance (historical biometric data) under similar conditions (device settings such as speed, inclination). In such an example, the analyzer may refer to the user profile to review the historical data and compare such data with the newly measured biometric data. Accordingly, the analyzer may identify the difference in the biometric data. In another example, the predetermined criteria may be the user preferences, In such an example, the analyzer reviews the user preference associated with the biometric data, and determine how the biometric data matches with the user preference. In other examples, the predetermined criteria may be trend data. In such an example, the analyzer reviews the user's biometric data in view of what is considered normal for a healthy person. It should be noted that even though three examples are shared here, there may be many other similar predetermined criteria used by the analyzer.

At block 330, the analyzer determines a setting adjustment for the device based on the analysis of the biometric data. More specifically, the analyzer decides what needs to be changed in the device in view of the analysis performed in the previous block. For example, the analyzer considers the change in the biometric data of the user (e.g., increase in body temperature) and determines how the device speed needs to be adjusted for the safety of the user. In one example, the analyzer may refer to the user profile to review the historical biometric data for the user or the user preferences, and determine what device setting adjustment is necessary. In one implementation, the setting adjustment may be selected from a list of predetermined setting adjustments. The predetermined setting adjustments may be previously associated with a certain biometric data change. For example, one of the predetermined setting adjustment may be reducing the device speed if the user's temperature has changed over 2 degrees Fahrenheit. Accordingly, when the biometric data change as a result of the analysis is an increase in the user's temperature by 3 degrees Fahrenheit, the analyzer automatically selects the setting adjustment of reducing the device's speed.

At block 340, the analyzer instructs the device to implement the setting adjustment. More specifically, the analyzer sends a command to the device to change its settings based on the way defined by the analyzer. For example, the analyzer instructs that the device should reduce its inclination by a certain amount.

The above discussion is meant to be illustrative of the principles and various embodiments of the present invention, Numerous variations and modifications will become apparent to those skilled in the art once the above disclosure is fully appreciated. It is intended that the following claims be interpreted to embrace all such variations and modifications.

What is claimed is:

1. A system for analyzing biometric data, comprising:
a sensor unit to measure biometric data of a user;
a controller unit coupled to the sensor to perform an analysis of the biometric data based on at least one predetermined criteria and determine at least one setting adjustment for a device based on the analysis; and
a communication unit to deliver an instruction from the controller to the device, wherein the instruction includes the at least one setting adjustment associated with the device.

2. The system of claim 1, further comprising a storage unit to store the biometric data.

3. The system of claim 1, wherein the device comprises a storage unit to store the biometric data.

4. The system of claim 1, wherein the biometric data comprises heart rate, pulse rate, temperature, respiration, acceleration, skin resistivity, muscle contractions and/or alike.

5. The system of claim 1, wherein the controller unit automatically selects a predetermined setting adjustment based on a list of predetermined setting adjustments associated with the user.

6. The system of claim 1, wherein the predetermined setting adjustments are generated based on user preferences, users historical biometric data and alike.

7. The system of claim 5, wherein the predetermined setting adjustment is unique to the user.

8. The system of claim 1, wherein the sensor unit, controller unit and the communication unit are in a mobile device such as a watch, band, mobile phone, tablet, phablet, implant or alike.

9. A processor-implemented method of analyzing biometric data, comprising:
measuring, by a sensor, biometric data of a user;
performing, by a controller, an analysis of the biometric data based on at least one predetermined criteria;
determining, by a controller, a setting adjustment associated with a device based on the analysis; and
instructing, by a controller, the device to implement the setting adjustment.

10. The method of claim 1, further comprising creating a profile for a user, the profile comprising data related to user preferences, user settings, user's prescribed targets, historical biometric data and/or alike.

11. The method of claim 1, wherein measuring the biometric data of the user further comprising measuring the biometric data continuously.

12. The method of claim 1, wherein performing the analysis of the biometric data comprises comparing the biometric data to historical biometric data associated with the user and determining a change in the biometric data.

13. The method of claim 12, wherein determining the setting adjustment further comprises selecting the setting adjustment based on the change in the biometric data.

14. A non-transitory computer-readable medium comprising instructions which, when executed, cause a system to:
measure biometric data of a user;
perform an analysis of the biometric data based on at least one predetermined criteria;
determine a setting adjustment associated with a device based on the analysis; and
instruct the device to implement the setting adjustment.

15. The non-transitory computer-readable medium of claim 11, further comprising instructions when executed cause the system to store the biometric data in a storage unit.
